(19) United States
(12) Patent Application Publication
(10) Pub. No.: US 2024/0070327 A1
(43) Pub. Date: Feb. 29, 2024

(54) PLATFORM FOR SHARING ATHLETE DATA

(71) Applicant: LINKSPORTS INTERNATIONAL CORP., Toronto (CA)

(72) Inventor: Neissan MONADJEM, Toronto (CA)

(21) Appl. No.: 18/260,118

(22) PCT Filed: Dec. 31, 2021
(86) PCT No.: PCT/CA2021/051907

§ 371 Date: Jun. 30, 2023

Related U.S. Application Data
(60) Provisional application No. 63/132,671, filed on Dec. 31, 2020.

(51) Int. Cl. G06F 21/64; G06Q 20/02; G06Q 20/36; H04L 9/00

(52) U.S. Cl. CPC G06F 21/64; G06Q 20/02; G06Q 20/3676; H04L 9/50

(57) ABSTRACT

A computer-implemented method for sharing, on a computer-implemented data-sharing platform, athlete data of users, comprising: issuing, via a transaction module, digital tokens for acquisition by the users, each digital token having a monetary value and purchasable by the users via the transaction module or from a third party entity communicatively coupled to the transaction module; receiving, via a data intake module, the athlete data from a user; assigning public rewards to the user based on the athlete data received, the public rewards visible to all of the users of the platform; verifying the athlete data by one or more third party verifiers and/or by cross-reference to verifying data received from the user; and sending at least a portion of the verified athlete data to a distributed ledger. Each of the users of the platform has a digital wallet on the distributed ledger for maintaining a balance of the digital tokens.

### FIELD

The following relates generally to computer-implemented methods and systems for acquiring, verifying and sharing athlete data, and in particular, computer-implemented methods and systems for acquiring, verifying and sharing athlete data using rewards-based incentivization for data sharing and verification.

### BACKGROUND

Current data-sharing regarding athletic skills of amateur athletes, and those aspiring to become professional athletes, in various sports (such as soccer, basketball, football, baseball, hockey, or cricket) tends to be limited to self-published and unverified information, typically on different social media platforms. There is no universal database of pre-professional athletes which incentivizes the use of verifiable data, and so the current systems tend to be unverified, disparate, and/or inherently biased since they depend on the incumbent player as a single source of the information.

Further, while there are methods for collecting data from an activity which exist today, most are time-consuming and costly due to individual data collectors traversing areas of interest to capture and collect data. Some data collection efforts by scouts and coaches of various sports have turned to crowdsourcing to reduce costs and have access to a larger pool of players. However, issues still arise concerning questions about data quality and gaps in data collection. It is difficult to cause a pool of data curators to work in unison and collect data in a structured manner that traverses all areas of interest (such as various types of sports), and which collects all types of data of interest. Further, it is difficult to determine the standards of evaluation to apply on all data of interest that has been captured and stored.

The discussion of the background herein is included solely to explain the context of the invention(s) described herein, and is not to be taken as an admission that any of the referenced material was published, known, or part of the common general knowledge as of the priority date of any of the claims herein.

### SUMMARY

In accordance with an aspect, there is provided a computer-implemented method for sharing, on a computer-implemented data-sharing platform, athlete data of users of the platform. The method comprises: issuing, via a transaction module of the platform, digital tokens for acquisition by the users of the platform, each of the digital tokens having a monetary value and purchasable by the users via the transaction module or from a third party entity communicatively coupled to the transaction module; receiving, via a data intake module of the platform, the athlete data from a user of the platform; assigning public rewards to the user based on the athlete data received, the public rewards visible to all of the users of the platform; verifying the athlete data by one or more third party verifiers and/or by cross-reference to verifying data received from the user via the data intake module; and sending at least a portion of the verified athlete data to a blockchain, the platform comprising the blockchain. Each of the users of the platform has an associated digital wallet on the blockchain for maintaining a balance of one or more of the digital tokens.

In accordance with another aspect, there is provided a computer-implemented method for sharing, on a computer-implemented data-sharing platform, athlete data of users of the platform. The method comprises: issuing, via a transaction module of the platform, digital tokens for acquisition by the users of the platform, over a network via a user computing device, each of the digital tokens having a monetary value and purchasable by the users over the network via the transaction module or from a third party entity communicatively coupled over the network to the transaction module via a third party computing device; receiving, via a data intake module of the platform, the athlete data from a user of the platform via the user computing device over the network; assigning, via the transaction module, public rewards to the user based on the athlete data received, the public rewards visible to all of the users of the platform; verifying, via a verification module of the platform, the athlete data by one or more third party verifiers and/or by cross-reference to verifying data received from the user via the data intake module; and sending, over the network, at least a portion of the verified athlete data to a distributed ledger, the platform comprising the distributed ledger. Each of the users of the platform has an associated digital wallet on the distributed ledger for maintaining a balance of one or more of the digital tokens.

In accordance with yet a further aspect, there is provided a system for sharing athlete data of users of the system, the system comprising: one or more platform computing device(s), each of the platform computing device(s) comprising non-transient computer-readable memory, a communication module, a transaction module, a data intake module, a verification module, and at least one processor communicatively coupled to the memory, the communication module, the transaction module, the data intake module, and the verification module; and a distributed ledger. The memory has stored thereon or therein computer-executable instructions for: issuing, via the transaction module, digital tokens for acquisition by the users over a network via respective user computing devices, each of the digital tokens having a monetary value and purchasable by the users over the network via the transaction module or from a third party entity communicatively coupled over the network to the transaction module via a third party computing device and the communication module; receiving, via the data intake module, the athlete data from a user of the system, over the network via a user computing device and the communication module; assigning, via the transaction module, public rewards to the user based on the athlete data received, the public rewards visible to all of the users of the system; verifying, via the verification module, the athlete data by one or more third party verifiers and/or by cross-reference to verifying data received from the user via the user computing device, over the network, via the communication module and the data intake module; and sending, over the network, at least a portion of the verified athlete data to the distributed ledger via the communication module. Each of the users of the system has an associated digital wallet on the distributed ledger for maintaining a balance of one or more of the digital tokens.

### BRIEF DESCRIPTION OF THE DRAWINGS

Aspects of the application will now be described by way of example only with reference to the appended drawings in which:

FIG. 1 depicts a schematic diagram of an example platform system described herein;

FIG. 2 depicts a schematic diagram of an example computing device described herein;

FIG. 3 depicts a schematic diagram of an example platform described herein;

FIG. 4 depicts a flow diagram of an example method described herein;

FIG. 5 depicts an example method for acquiring digital tokens described herein;

FIG. 6 depicts a schematic diagram of example athlete data described herein;

FIG. 7 depicts a schematic diagram of example sponsors described herein;

FIG. 8 depicts a flow diagram of another example method described herein;

FIG. 9 depicts a flow diagram of yet another example method described herein;

FIG. 10 depicts a schematic diagram of an example score card described herein;

FIG. 11 depicts a schematic diagram of another example score card described herein; and

FIG. 12 depicts a schematic diagram of an example screen shot of a video tutorial described herein.

### DETAILED DESCRIPTION OF CERTAIN ASPECTS

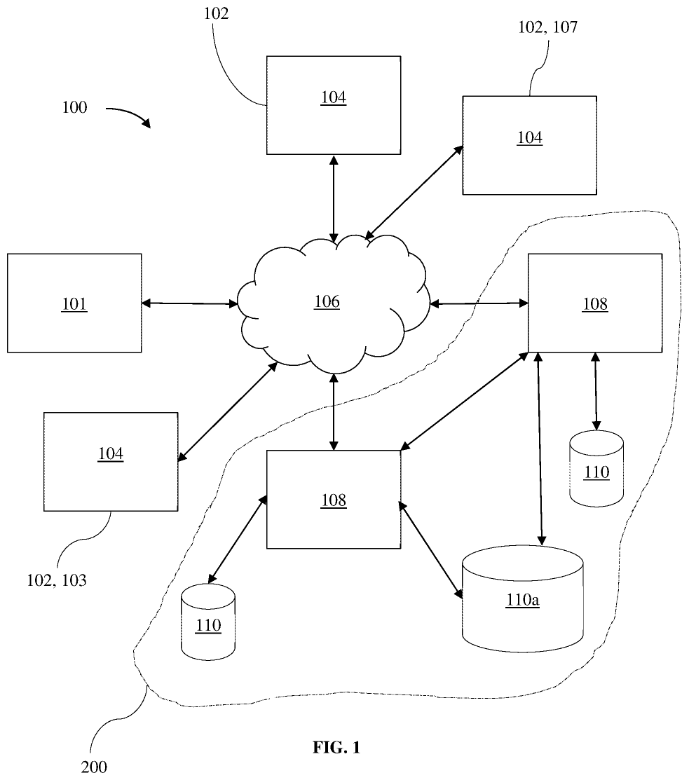

With reference to the figures, the presently described aspects pertain to a computer-implemented method for sharing, on a computer-implemented data-sharing platform or system 200 (such as a software as a service (SaaS) platform), athlete data 500 of users 102 of the platform 200. Each user 102 may be required to pay a monthly subscription fee to access the platform 200 (or some users 102 may access the platform for free for access to a more limited feature-set, and/or there may be tiers of feature access corresponding to tiers of subscription fees). Each user 102 (and/or other interfacing entities or components, such as entities or components 101, 103, 107) may access the platform 200 via a user computing device 104 (which itself may comprise an interfacing entity or component of the platform 200) capable of accessing the platform 200 by known communications protocols over a network 106, such as the Internet 106. The user computing device 104 may comprise any networked user computing device, such as a tablet, smart phone or computer (desktop or laptop), for example. The platform 200 may be implemented by one or more platform computing device(s) (e.g., computers or servers) 108 communicatively coupled to one or more data stores or databases 110 storing platform data, which may be stored in a distributed and synchronized manner across multiple databases 110. The databases 110 may comprise a distributed ledger technology (“DLT”) or distributed ledger (or “DL”, such as, but not necessarily limited to, a blockchain) 110a, such that the platform 200 comprises the DL 110a. Any use of the term “blockchain” herein is intended to encompass any suitable DLT or DL, including DLTs based on chains of blocks, and DLTs not based on chains of blocks (such as a Directed Acyclic Graph (DAG) DLT). The platform 200, and the users 102, including their computing devices 104 for accessing the platform 200, as well as third party entities 101 communicatively coupled to the platform 200, and the network(s) 106 over which the platform 200 is accessed, together comprise a platform system or ecosystem 100. While FIG. 1 depicts the platform 200 as being separate from the interfacing entities or components 101, 102, 103, 104, it will be appreciated that client-side instance(s) of the platform 200 (such as a user interface 202, 204 of the platform 200), served by the platform computing device(s) or server(s) 108 over network(s) 106 to the interfacing entities or components 101, 102, 103, 104 for access on computing devices 104, may also comprise a part of the platform 200.

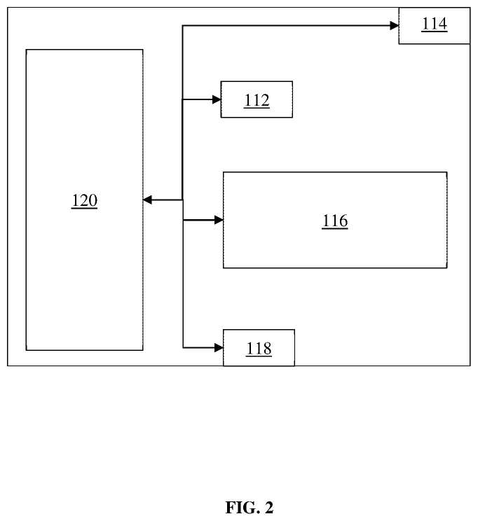

With reference to FIG. 2, any of the computing devices 104, 108 described herein may comprise memory 112, a communication module 114, a display 116, one or more input devices 118, and at least one processor 120 communicatively coupled to the memory 112, communication module 114, display 116, and input device(s) 118. The memory 112, which may comprise non-transient computer-readable memory 112, may comprise or have stored thereon or therein computer-executable instructions that when executed by the processor(s) 120 carry out the method step(s) described herein, and may comprise one or more of a local and/or remote hard disk or hard drive, of any type, ROM (read-only memory) and/or RAM (random-access memory), buffer(s), cache(s), flash memory, optical memory (e.g., CD(s) and DVD(s)), and any other form of volatile or non-volatile storage medium in or on which information may be stored for any duration. The communication module 114 may enable the computing devices 104, 108 to communicate with one or more other components of the platform system 100 via a wired and/or wireless communication network 106. The network 106 may comprise a direct link between communicating components of the platform system 100 (including among components of the platform 200), or an indirect one, including but not limited to communication by Ethernet™ Bluetooth™, WiFi™, WiMAX™ (fixed or mobile), and any suitable cellular communications protocols including, but not limited to up to at least 5G protocols, such as GSM, GPRS, EDGE, CDMA, UMTS, LTE, LTE-A, and IMS, for example, and any other communications protocols suitable for the method(s), system(s) and device(s) described herein, including any proprietary protocols. The network 106 may comprise a single network or more than one interconnected network, of any type suitable for the method(s), system(s) and device(s) described herein, including but not limited to wired or wireless LANs (local area networks), WANs (wide area networks), MANs (metropolitan area networks), mesh or ad hoc networks, VPNs (virtual private networks), the Internet, and any other suitable network type, in any suitable network configuration or topology (e.g., mesh, token ring, tree, star, etc.), and any suitable combination of the foregoing. Although not shown in FIG. 1, the platform system 100 may further include any components necessary to effect the communication and/or network type(s) used, and may also include components for increased network security, for example, access points, routers, and firewalls.

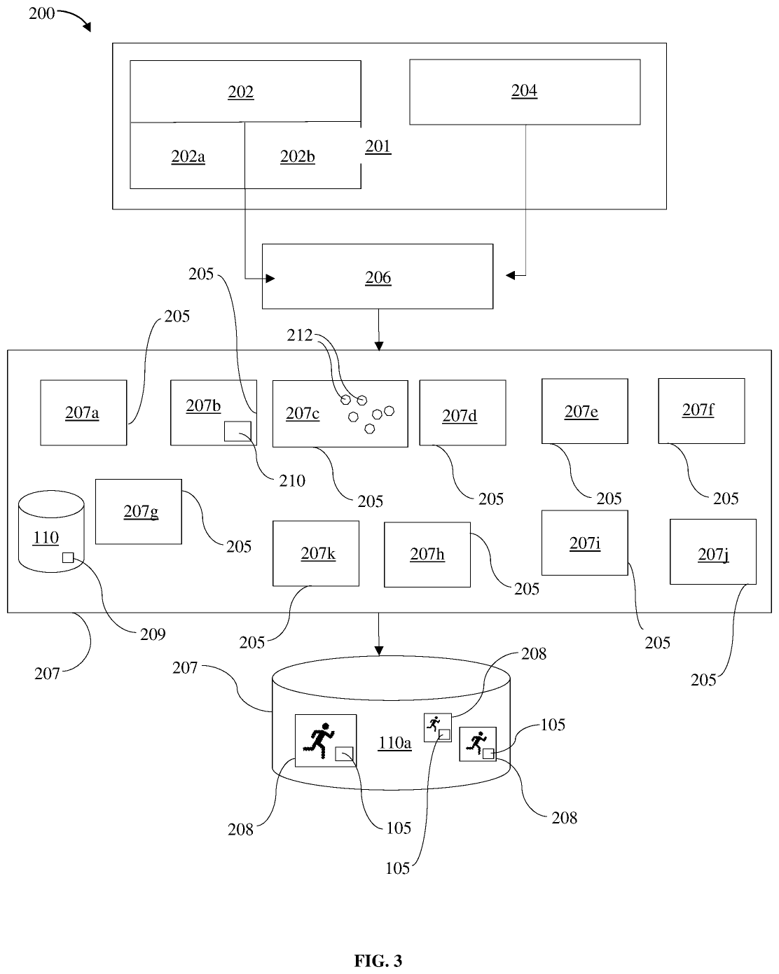

With reference to FIG. 3, a high-level diagram of an example platform 200 for carrying out method step(s) described herein may comprise a user interface layer 201. The user interface layer may comprise a user interface (UI) 202 (and in some aspects, the UI 202 may comprise a regular user interface or login 202a and a premium user interface or login 202b), and a professional user interface 204. The platform 200 may further comprise an application management layer 206 for managing data flow between the user interface layer 201 and the platform backend 207 of the platform 200 (the platform backend 207 in some aspects also comprising the DL 110a). Additionally or alternatively, the application management layer 206 may contain the logic for directing the user 102 to the correct platform UI 202, 204 based on verification, by a login verification module 207a, of the user's login credentials and access rights for the user 102 based on cross reference of the login credentials against system database(s) 110.

The professional UI 204 may be accessed by sports associations or clubs, medical professionals or other sports or athletic performance authorities or partners of the platform 200. The regular UI 202 may be accessed by athletes or everyday visitors of the platform 200. As described above, the regular UI 202 may have further divisions, such as for a premium user interface 202b (which may be for users 102 with paid subscriptions to the platform 200) and a standard or regular user interface 202a (which may be for users 102 with free subscriptions to the platform 200, or subscriptions with a lower fee than that of premium users 102). As shown in the example of FIG. 3, the platform backend 207 may comprise, e.g., various processing modules 205 (each comprising (i) computer-executable instructions stored in or on non-transient computer-readable memory 112 for execution by processor(s) 120 of platform computing device(s) 108 to carry out method step(s) described herein, and/or (ii) computing device(s) 108 (as described herein) for execution of such computer-executable instructions by processor(s) 120 thereof), such as one or more of the login verification module 207a, data intake module 207b, transaction module 207c (which may, e.g., issue rewards 212 (such as points and digital tokens 212 (described further below)), and/or allow for tokens 212 or rewards 212 to be redeemed, such as for goods or services offered through the platform 200, or for fiat currency or cryptocurrency, as further described below), service provider accreditation module 207d, verification module 207e, extraction/reporting module 207f, goods and services module 207h, score card generation module 207i, ranking module 207j, search module 207k, and artificial intelligence module and/or machine learning (AI) architecture 207g (which may employ artificial intelligence and/or machine learning to carry out any of the method steps described herein by some or all of the processing modules 205). In some aspects, the processing modules 205 may transmit or send some or all data verified by the verification module 207e (which, as described above, may use the AI module 207g to carry out some or all verification of data received via the data intake module 207b) to the DL 110 as DL data 208.

The data intake module 207b may perform processes or algorithms to populate a user's 102 profile with data to be verified by the verification module 207e. This data may be uploaded by the user 102 as they create a user profile or update their user profile for the platform 200. Other parties, such as professional users 102, may provide data for members of their associations or clubs in order to update those members who have user profiles 102 on the platform 200. The data intake module 207b may save the data inputted by users 102 to database(s) 110 where it awaits verification, such as by the verification module 207e. Data that may be inputted into the platform 200 may include, but is not necessarily limited to, statistics, highlights, technical information, physiological information, video, photos, contracts, and other sports-related or user-related data. In some aspects, the data intake module 207b may also automatically search social media or other public sites for a user's 102 sports-related or user-related data, once the user 102 has created their profile, and as described above, the data intake module 207b may use the AI module 207g to carry out such automated online data acquisition and parsing to generate auto-generated data. The data intake module 207b may use the AI module 207g to classify, such as by assigning classification attributes or metadata to, the user-inputted or auto-generated data (collectively, the “data”) before saving the data to the database(s) 110. Additionally or alternatively, the user 102 may provide the information required for proper classification at the time of initial user profile creation, or thereafter when updating the user profile. Classification may be by player identification, sport, action, and/or other sports statistics. The AI module 207g may discard any unusable videos or photos uploaded to the data intake module 207b that do not meet a minimum quality requirement or threshold. The AI module 207g may also flag any corrupted, inconsistent or otherwise unusable data for further auditing and/or human verification, or may discard such data and accordingly notify the user 102.

The verification module 207e may verify data taken in or received by the data intake module 207b. Verification of the data allows it to become certified or verified data, and some or all of the verified data may be stored on the blockchain or other DLT 110a, as described in further detail, below. Data may be verified by photographic evidence, video evidence, surveys, third party verifiers, statistics, contracts, and other forms of documentation (e.g., tournaments claimed by a user 102 to have been won may be verified by posting a picture of the tournament trophy or a statement from the tournament organizers, for example). The verification module 207e may employ the AI module 207g to verify photographic and video evidence through facial recognition, for example. Verification may be also manually conducted by third party verifiers 103, such as other users 102, former teammates of a user 102, or testimonials or confirmations provided by authorities such as coaches, educational institutions, sports teams, or sports associations associated with a user 102, for example. Such third party verifiers 103 may also be users 102 of the platform 200, in order to carry out their verifications on the platform 200.

The verification module 207e may verify data uploaded by users 102 and transmit or send the verified data to the DL 110a, only after such data is attested to by one of the categories of third party verifiers 103 or verifier users 103, 102 of the platform 200. Third party verifiers 103 may comprise athletic departments of educational institutions, sports clubs, physiotherapists or physical therapists, doctors, and/or other sporting or sports-related institutions or professional service providers having experience with the user 102 as an athlete. Third party verifiers 103 that are professional service providers may be accredited as such by the service provider accreditation module 207d of the backend 207, which may employ the AI module 207g and public sources of information to accredit the service provider as a professional service provider, and as described in further detail below, the platform 200 (such as via the transaction module 207c) may attribute a higher point valuation to such professional verifier users 102 for their verifications than to non-professional verifier users 102 (e.g., visitors to the site, friends, family, or teammates of a user 102) for their verifications, as further described below.

Contrary to the sometimes complex analytics associated with professional athlete data (for example, in professional basketball, there is the Hollinger Assist Ratio, Pomeroy Assist Ratio, and Assists per Team Possession, just to name a few of the more advanced statistics tracked by analysts), the platform 200 is expected to allow for simplified, more readily or universally available data for amateur or non-professional athletes (including video footage of such athletes' sports play) to be uploaded to the platform 200 to be analyzed, including by AI/ML, in order to parse therefrom meaningful information to assist sports recruiters, such as scouts, in identifying new talent. It is expected that this will provide a platform 200 to amateur and non-professional athletes the world over to be seen, especially in countries where such visibility by sports recruiters tends to otherwise be difficult to achieve.

The transaction module 207c may assess and assign a value of rewards (which may comprise, e.g., points 212, and/or digital tokens 212 redeemable for fiat currency or cryptocurrency) to each third party verifier 103 for each verification, and/or for each block of verified data added to the DL 110a, as described in further detail below. The rewards value may be calculated based on the level of verification of the data (e.g., by professional verifier users vs. non-professional verifier users), for example, and/or any other relevant factors. Points may also be awarded for user rankings, polls completed by users 102, and/or other contests that may be generated on the platform 200, such as by other users 102 or sponsors 107 (discussed further below). Points may be accumulated and applied to a user's 102 ranking 602 in the platform 200 which, in turn, may affect a user's visibility on the platform 200 to other users 102 and/or sports recruiters (which may comprise professional users 102 of the platform 200). Points may, in some aspects, be used to purchase digital tokens 212, such as via the transaction module 207c.

The extraction/reporting module 207f may extract information from the database(s) 110, 110a, and display or compile into a report, a representation of the extracted information, such as upon request by a user (which may comprise, e.g., a professional user) 102 or an administrator of the platform 200. The extraction/reporting module 207f may, e.g., extract information from the non-DL data 209 and/or the DL data 208 from the database(s) 110, 110a to compile into a report which may be viewed by, e.g., professional users 102 of the platform 200. Whether reports are available, or the level of reporting available, from the extraction/reporting module 207f, may depend on whether the user 102 is a standard/regular user 102, premium user 102, or a professional user 102.

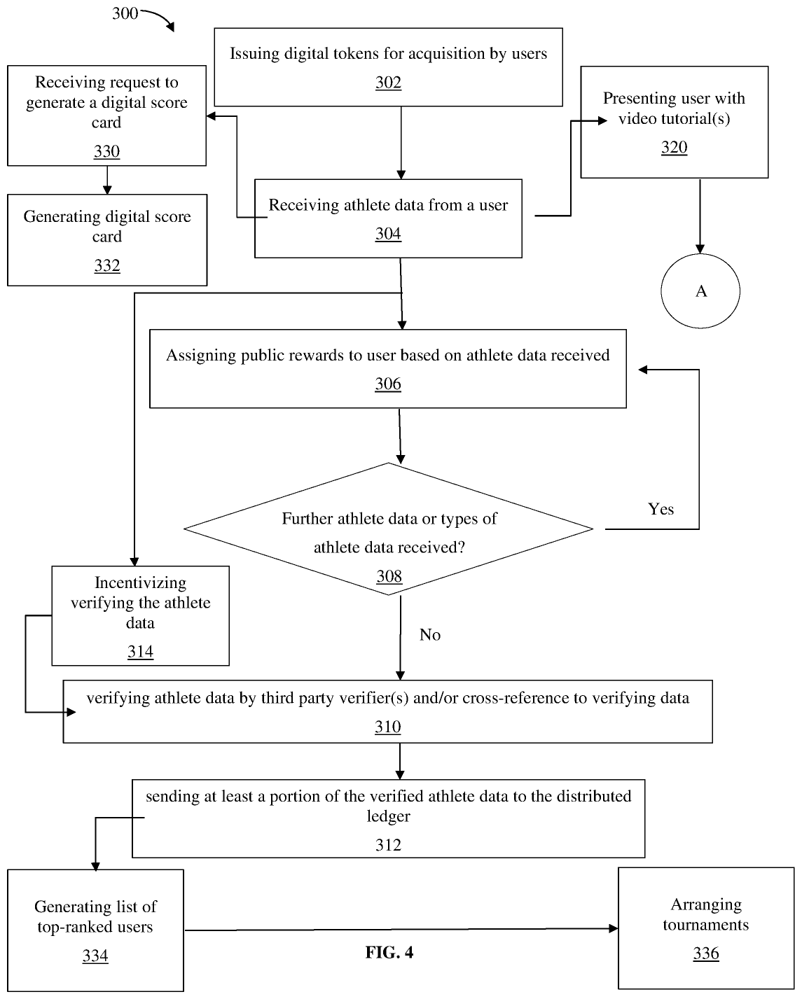

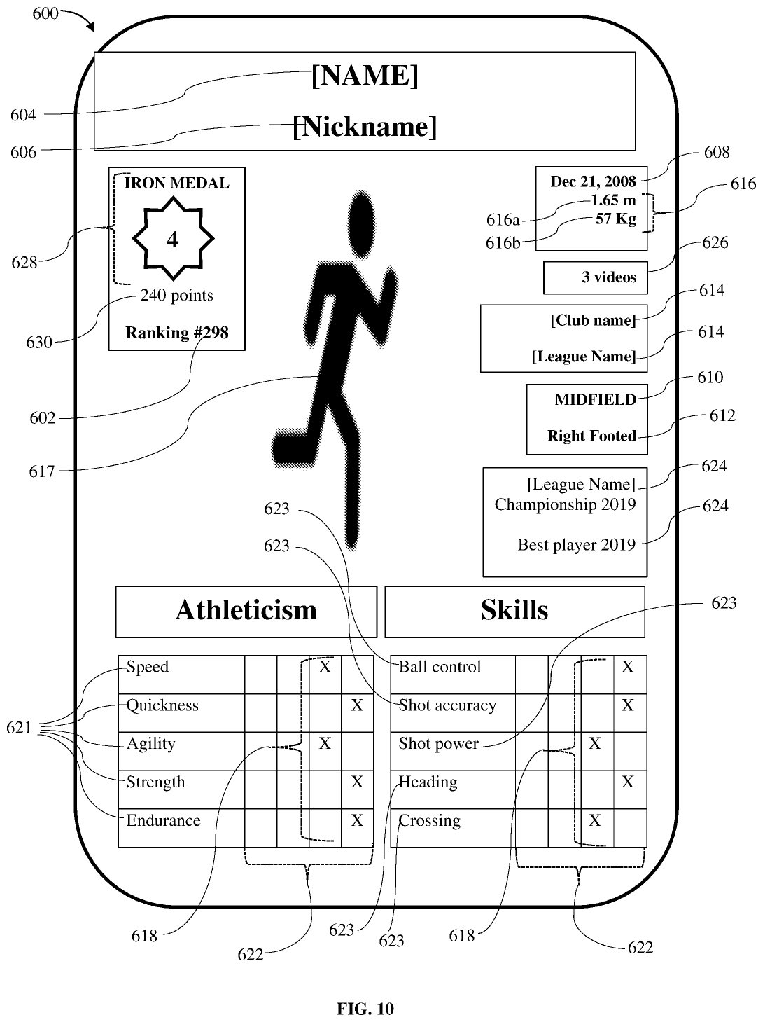

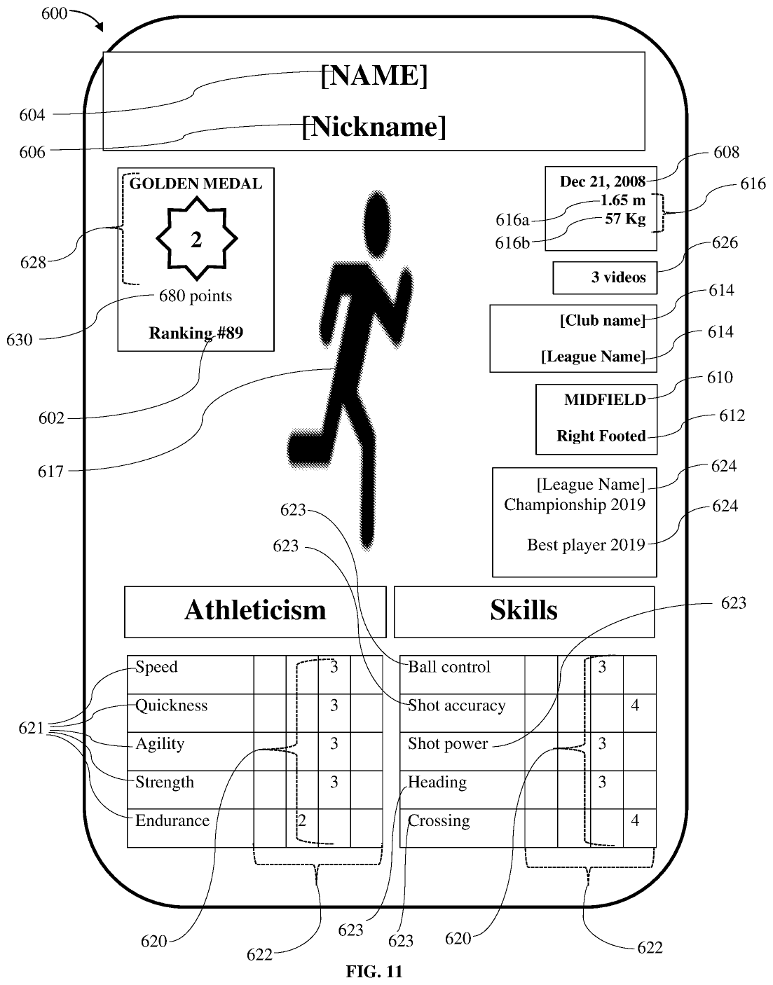

With reference to FIG. 4, in some aspects there is provided a computer-implemented method 300 for sharing, on a computer-implemented data-sharing platform 200 (such as a software as a service (SaaS) platform 200), athlete data 500 of users 102 of the platform 200. The method 300 may, in some aspects, comprise: issuing 302, such as via a transaction module 207c of the platform 200, digital tokens 212 for acquisition by the users 102 of the platform 200, such as over a network 106 via a user computing device 104. Each of the digital tokens 212 may have a monetary value, and may be purchasable by the users 102 (such as over the network 106) via the transaction module 207c, or from a third party entity 101 (e.g., cryptocurrency exchanges, such as Coinsquare™ or Coinbase™) that is communicatively coupled (such as over the network 106) to the platform 200, such as to the transaction module 207c of the platform 200 (such as via a third party computing device 104). The method 300 may further comprise: receiving 304, such as via the data intake module 207b of the platform 200, the athlete data 500 from a user 102 of the platform 200 (such as via the user computing device 104 over the network 106); and assigning 306 (such as via the transaction module 207c) public rewards 212 to the user 102 based on the athlete data 500 received (such as via the data intake module 207b). In some aspects, the public rewards 212 may be visible to all of the users 102 of the platform 200. Further, the public rewards 212 may comprise points 212, virtual badges or medals 628 (see, e.g., FIGS. 10 and 11, described further below) of ascending levels corresponding to respective ascending amounts of the points 212, or both. The assigning 306 may comprise, e.g., assigning 306 further public rewards 212 to the user 102 for any additional athlete data 500 received from the user 102 after the initially received 304 athlete data 500. That is, as shown at step 308 of the method 300, assigning 306 the public rewards 212 based on the athlete data 500 received may comprise adjusting an amount of the assigned public rewards 212 (or further assigning 306 public rewards 212) to be commensurate with an amount of the athlete data 500 received (such as via the data intake module 207b) and/or an amount of the types of the athlete data 500 (e.g., text, video) received (such as via the data intake module 207b).

In some aspects, the method 300 may further comprise: verifying 310 (such as via a verification module 207e of the platform 200) the athlete data 500 by one or more third party verifiers 103 and/or by cross-reference to verifying data (such as photos or scanned documents) received from the user 102 via the data intake module 207b (such as at step 304); and sending 312 (such as over the network 106) at least a portion of the verified athlete data 500 to the DL 110a of the platform 200. As shown in FIG. 3, each of the users 102 of the platform 200 may have an associated digital wallet 105 on the DL 110a for maintaining a balance of one or more of the digital tokens 212. Each digital wallet 105 may be previously created for the respective user 102 by a third party cryptocurrency exchange. Alternatively, the platform 200 may comprise a centralized exchange for the secure storage of the funds (e.g., tokens 212) of each digital wallet 105.

With reference to FIG. 3, in some aspects, the data intake module 207b may comprise a data input interface 210, which may be communicatively coupled to the input device(s) 118 of the computing device(s) 104, 108, such as for receiving 304 the athlete data 500 via input(s) of the user(s) 102, via the user computing device(s) 104, to the data input interface 210. The data input interface 210 may be configured to receive any suitable form or type of data, such as text (e.g., by way of text entry field(s)), document(s) of any suitable type (e.g., by way of document upload interface(s)), video recordings of any suitable type (e.g., by way of video upload interface(s)), which may be communicatively coupled to a video capture input device 118 (e.g., a camera 118), such as of a user computing device 104), etc.).

The DL 110a may comprise a blockchain 110a, which may comprise any suitable form of blockchain for use in, or with, the platform 200, including a proof-of-work (PoW) or proof-of-stake (PoS) based blockchain 110a. In some aspects, the blockchain 110a may comprise a permission-based blockchain 110a (i.e., a blockchain 110a without anonymous nodes), and sending 312 the at least the portion of the verified athlete data 500 to the blockchain 110a may comprise validating the at least the portion of the verified athlete data 500 as a block transaction on the blockchain 110a by a proof-of-stake validation. By using a proof-of-stake blockchain 110a, it is expected that the validation of a new block of information on the blockchain 110a will not require the heavy computational costs that tend to be involved in the mining operations associated with proof-of-work blockchains, and further, avoid the need to generate cryptocurrency (e.g., the digital tokens 212 issued by the transaction module 207c) to reward such mining operations. Rather, the platform 200 (such as via platform administrators and/or valuable members of the platform ecosystem including, e.g., founders, first partnered independent sports institutions and the largest asset/digital token holders as shareholders, investment funds, and/or advisors) may hold enough coins or digital tokens 212 to vote to authenticate a new generated block of a player's 102 athlete data 500.

In some aspects, the digital tokens 212 may be sellable to, or redeemable via, the third party entity(ies) 101 or the transaction module 207c to realize the monetary value of the digital tokens 212 as fiat currency (such as the Canadian or U.S. dollar) and/or cryptocurrency (such as a cryptocurrency used outside of the platform 200, such as Bitcoin™), for example. For example, a user 102 may purchase digital token(s) 212 via the transaction module 207c, and later sell some or all of the digital token(s) 212 back to the platform 200 for circulation to further users 102, via the transaction module 207c, and so any user 102 (whether having a regular account, premium account, or professional account) may use the platform 200 as a means for investment, buying and selling the platform 200 digital token(s) 212 in an effort to make a monetary gain, much like stocks that are purchased and sold via a public stock market or exchange.

The digital tokens 212 may initially have a nominal valuation set by the platform 200 administrators (and as a reference, the value of a digital token 212 may be based, e.g., on a combination of fiat currencies (such as a combination of the Swiss Franc, Euro, Yen, Dollar, and Brazilian Real) or, e.g., the value of 1/100th of a gram of a precious metal that is traded globally). Thereafter, the digital tokens 212 may be released to be publicly traded and to acquire a free market value; i.e., the platform 200 may also allow for an after-market trading space for the digital tokens or coins 212 issued by the transaction module 207c, using present coin-exchange and trading institutions 101, to create a trading market for the digital coins or tokens 212, which may be called “Sportscoins” or “Sportscash”, for example. This is expected to allow for some level of speculation as the digital tokens or coins 212 are released into the market with a fungibility allowed by conversion of the digital tokens to regulated fiat currency or legal tender, such as the U.S. Dollar, the Canadian Dollar or the Brazilian Real, such as through the transaction module 207c and/or a third party bank or exchange 101, for example. In this way, the digital tokens 212 issued by the transaction module 207c may act not only as a utility coin, but also as means of exchange, as trust in the digital tokens 212 in the market increases its negotiable value in the trading space. As the platform 200 demonstrates its effectiveness for sharing athlete data 500 of users 102, in order to yield successful sports contracts and fruitful sponsorships on the platform 200 (as described further, below), as well as through the demonstration of sound business principles and administration of the platform 200, as evidenced through the capacity of the platform 200 for steady and sustainable growth (such as through the platform's 200 scalable infrastructure), trust in the digital tokens 212 is expected to increase in the marketplace which, combined with sound management of the platform 200, may increase the digital token's trading value. Unlike speculative cryptocurrency issued in initial coin offerings (ICOs) based on whitepapers, the platform's 200 digital tokens 212 would not be used as a security to generate capital in a crowdfunding manner. Rather, the digital tokens or coins 212 issued by the transaction module 207c of the platform 200 are expected to attain value through the effectiveness of the platform 200 as a marketplace and social network for sports disintermediation, allowing amateur or pre-professional athletes 102 to be more easily discovered and to more readily share their pertinent data or athlete data 500 with sports recruiters 102 while securing sponsorships to assist in that endeavor, as discussed further below.

In some aspects, the digital tokens 212 may be redeemable by a user 102, via the transaction module 207c, to (i) upgrade an account level of the user 102 (such as from a regular user 102 to a premium user 102 (the premium account may, e.g., increase visibility of the user 102 on the platform 200 to other users 102 and/or to professional users 102 (such as sports recruiters and scouts)) and/or (ii) to purchase goods (e.g., sporting attire, such as shoes offered at a sponsored price) and/or services (e.g., an online personal training coach, or online courses (e.g., athletic training programs (e.g., “Tactical Football Hints for Beginners”), or courses covering topics generally pertinent to athletes, such as courses about sports nutrition or professional negotiations with sports agents, for example)) offered on or through the platform 200, such as via the goods and services module 207h of the platform 200.

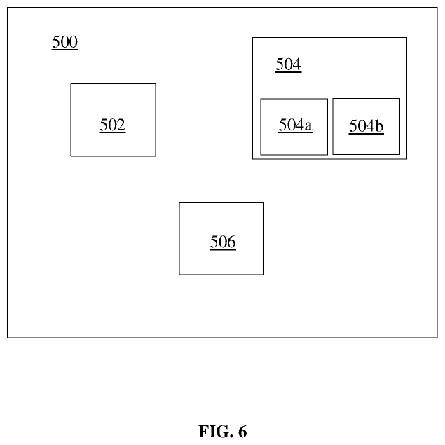

With reference to FIG. 6, in some aspects, the athlete data 500 may comprise: (i) non-athletic data 502; (ii) athletic data 504 comprising at least athleticism data 504a and skills data 504b; and/or (iii) promotional content data 506. With reference to FIG. 10 (depicting an example score card 600, described in further detail below), the non-athletic data 502 may comprise, e.g., the user's 102 name 604, nickname 606, birthday 608, gender (not shown), sport(s) played (e.g., “soccer”) (not shown, although inferred by reference to the “Club Name” 614 and “League Name” 614), position(s) played 610 (which may auto-populate with suitable options for the sport(s) selected by the user 102 as the user's 102 sport(s) played), laterality 612 (i.e., are they left-handed/footed or right-handed/footed), associated sports organizations 614 (e.g., where is the user playing?), anthropometric data 616 (including, but not necessarily limited to, the user's 102 height 616a and weight 616b), picture(s) 617 (such as profile picture(s) 617 showing the user's 102 face and/or action picture(s) 617 showing the user 102 playing a sport; see, e.g., FIGS. 10 and 11, depicting action pictures 617 of the user 102), and/or a list of and contact information for the third party verifier(s) 103 (not shown). The athleticism data may comprise information regarding athleticism, e.g., user self-assessment(s) 618 (or, as shown on the example score card 600 of FIG. 11, assessments 620 generated based on verified athlete data 500 rather than the non-verified self-assessments 618 shown in FIG. 10)) of athletic traits 621, such as speed 621, quickness 621, agility 621, strength 621, and/or endurance 621, and the skills data may comprise information regarding athletic skills, e.g., user self-assessment(s) 618 of skill(s) 623 related to sport(s) played by the user 102 (i.e., skills that are sport-dependent, such as “ball control” 623, “shot accuracy” 623, “shot power” 623, “heading” 623, and “crossing” 623 for soccer). The skill(s) 623 may auto-populate with suitable options for the sport(s) selected by the user 102 as the user's 102 sport(s) played. The assessments 618, 620 may comprise a numbered scale (e.g., from 1 (poor—the leftmost assessment boxes 622 under “Athleticism” and “Skills” in FIGS. 10 and 11) to 4 (excellent—the rightmost assessment boxes 622 under “Athleticism” and “Skills” in FIGS. 10 and 11)) that may be entered by the user 102 or third party verifiers 103 via the data input interface 210 for the assessments 618 and 620, respectively (or, e.g., the assessments 620 may be generated via the verification module 207e based on the verified athlete data 500). The promotional content data 506 may comprise, e.g., athletic achievements data 624, such as video footage of the user's athletic achievements (e.g., a video montage of the user's 102 best moments in sports, such as soccer highlights), which may be accessible via the digital score card 600 (in which case the user interface 202 may comprise the digital score card 600), such as via the video(s) indicator 626 shown in FIGS. 10 and 11. Additionally or alternatively, the video(s) indicator 626 may serve to indicate the number of videos associated with the user 102. The athlete data 502 may comprise any suitable type of data, including text, audio, video, and/or any other type of data suitable for conveying the athlete data 500, such as documents (e.g., image files, video files, document files (e.g., scanned documents), and the like.

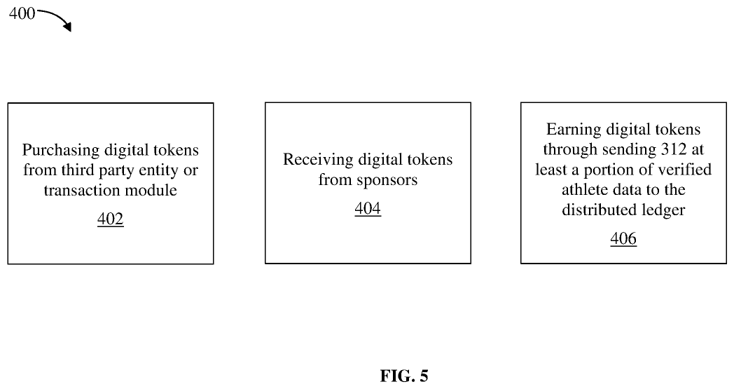

With reference to FIG. 5, in some aspects, the user 102 may acquire one or more of the digital token(s) 212 by method 400, which may comprise: (i) purchasing 402 the one or more of the digital tokens 212 from the third party entity 101 or the transaction module 207c; (ii) receiving 404 the one or more of the digital tokens 212 from one or more sponsors 107 (each sponsor 107 may be a user 102 of the platform 200), such as via the transaction module 207c; and/or (iii) earning 406, such as via the transaction module 207c, the one or more of the digital tokens through sending 312 at least the portion of the verified athlete data to the DL (e.g., blockchain) 110a, in which case the one or more digital tokens 212 acquired by the user 102 may comprise a user verification reward amount (i.e., a reward for the verification) of the digital tokens 212 (which amount may be determined by the transaction module 207c), such as in the user's digital wallet 105 on the DL (e.g., blockchain) 110a. In some aspects, the at least the portion of the verified athlete data 500 that is sent 312 to the DL (e.g., blockchain) 110a may comprise only the athletic data 504 (comprising at least the athleticism data 504a and the skills data 504b) and/or the promotional content data 506.

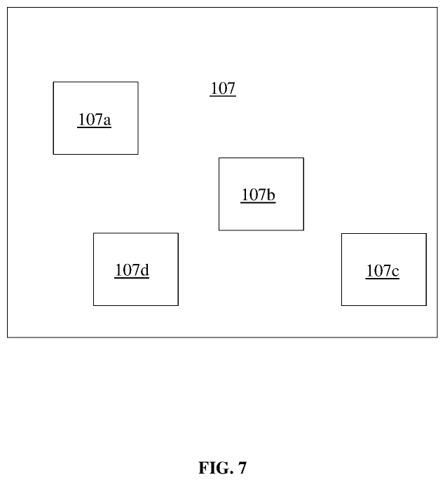

With reference to FIG. 7, in some aspects, the sponsor(s) 107 may comprise one of: (i) a personal or micro (PM) sponsor 107a, each PM sponsor 107a designating less than a first threshold amount of the digital tokens 212 to one or more PM-sponsored users 102 of the platform 200; (ii) a local or regional (LR) sponsor 107b, each LR sponsor 107b designating between the first threshold amount and a second threshold amount of the digital tokens 212 to sponsoring: (A) the platform 200, (B) one or more LR-sponsored users 102 of the platform 200, and/or (C) one or more LR-sponsored tournaments (which may be administered as a service via the goods and services module 207h) associated with the platform 200; and (iii) a national or global (NG) sponsor 107c, each NG sponsor 107c designating more than the second threshold amount of digital tokens 212 to sponsoring: (A) the platform 200, (B) one or more NG-sponsored users 102 of the platform 200, and/or (C) one or more NG-sponsored tournaments (which may be administered as a service via the goods and services module 207h) associated with the platform 200. In some aspects, the sponsors 107 may also comprise platform media sponsors 107d who do not acquire digital tokens 212, but advertise on the platform 200 for a fee paid to the platform 200, such as via the transaction module 207c of the platform 200. Sponsorship money entering the platform 200, such as via the transaction module 207c (e.g., through a bank institution 101 of which the sponsor 107 is a client and which transacts with the transaction module 207c, such as via a secure network connection over the network 106) may be converted by the transaction module 207c into the equivalent value of digital tokens 212 for use within the platform 200 ecosystem 100. In some aspects, whether a sponsor 107 is a PM sponsor 107a, LR sponsor 107b, or NG sponsor 107c may additionally, or alternatively, depend on the number of users 102 sponsored by the sponsor 107.

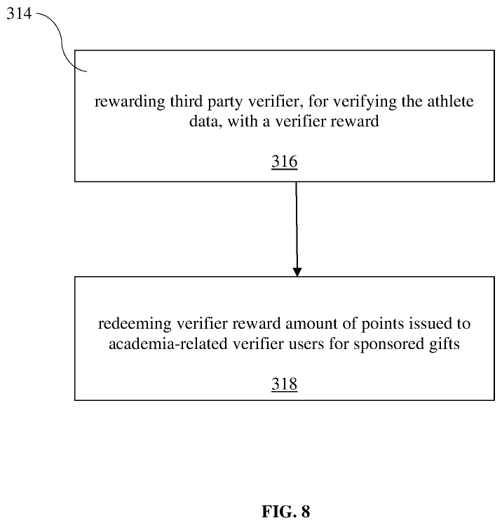

With reference to FIGS. 4 and 8, in some aspects, the method 300 may further comprise incentivizing 314 (such as via the transaction module 207c) verifying 310 the athlete data 500 by one or more third party verifiers 103 by rewarding 316 the one or more third party verifiers 103, for verifying 310 the athlete data 500, with a verifier reward 212 (i.e., as shown in FIG. 8, the incentivizing 314 may comprise the rewarding 316, although it will be appreciated that other forms of incentivizing 314 may be used). The verifier reward may, e.g., comprise (i) a verifier reward amount of the digital tokens 212 (which amount may be determined by the transaction module 207c) issued to professional verifier users 102 of the platform 200 (such as by the transaction module 207c), and/or (ii) a verifier reward amount of points (which amount may be determined by the transaction module 207c) issued to non-professional verifier users 102 of the platform 200 (such as by the transaction module 207c). This may mean, in some aspects, that the user 102 may require some funds (such as digital tokens 212 purchased by the user 102 via the transaction module 207c and/or digital tokens 212 granted to the user 102, such as by PM sponsors 107a (e.g., friends and family), so that the user 102 has funding available to issue verifier rewards in the form of digital tokens 212 to professional verifier users 102 as a reward for their verifications of the athlete data 500 (to the extent that such professional verifier users 102 are not rewarded by LR or NG sponsors, for example)). Each of the professional verifier users 102 may comprise a service provider accredited by a service provider accreditation module 207d of the platform 200 as a professional service provider (e.g., physiotherapists/physical therapists, trainers, doctors, performance advisors, etc.), and with an active account on the platform 200. Each of the non-professional verifier users 102 may not be accredited by the platform 200 (such as by the service provider accreditation module 207d) for providing professional services. The non-professional verifier users 102 may comprise, e.g., peers of the user 102 (e.g., current or former teammates), academia-related verifier users 102 of the platform (e.g., former school coaches and teachers), and/or other references provided by the user 102 (such as over the network 106 via the user computing device 104). Verification may be carried out by, e.g., a confirmation entered via the user interface 202 by professional or non-professional verifier users 102 and registered by the platform 200 (such as the verification module 207e thereof) as a verification of some or all of the athlete data 500. In some aspects, the platform 200 (such as the verification module 207e thereof) may not consider a portion of the athlete data 500 to be verified until a threshold number of verifications has been registered, and the threshold number of verifications may be with respect to each of the professional and non-professional verifier users 102 (i.e., two threshold values, one for each of the professional and non-professional verifier users 102), or with respect to both the professional and non-professional verifier users 102 (i.e., one threshold value, for the cumulative total of verifications received from both the professional and non-professional verifier users 102), or both. In some aspects, the platform 200 (such as the verification module 207e thereof) may only use the other references of the non-professional verifier users 102 as a source of verification as a last resort.

It is expected that the platform 200 may provide for a global (through a global platform 200) presence of PM sponsors 107a in the sports space, allowing for funding, akin to the principles of “crowd-funding”, although instead of large sums of money being directed to star athletes, the platform 200 is expected to allow for thousands of small investors or PM sponsors 107a, each providing (or betting, where they expect some return on their investment) a relatively small amount of money which is converted, such as by the transaction module 207c, into an equivalent value of digital tokens 212 or cryptocurrency 212, to sponsor their favorite young athletes' careers, while also allowing for larger investments from larger sponsors 107b, 107c.

When offered by friends and family 107a, these small donations or investments may help a user 102, such as a loved one of the PM sponsor 107a, to succeed in their journey to become a professional athlete. The donations or investments of a PM sponsor 107a may be converted to equivalent values of digital tokens 212, such as via the transaction module 207c, which are automatically transferred to a unique digital wallet 105 belonging to the specific user 102 for whom the donations/investments were earmarked or designated by the PM sponsor 107a via the user interface 202, 204. The user 102 may, in some aspects, only use the funds in their digital wallet 105 within the ecosystem 100 of the platform 200, such as to increase their visibility in the platform 200 or platform system 100 by, e.g., upgrading to a premium account, or to pay for goods and/or services (such as online courses or shoes offered at a sponsored price, as described above), such as via the transaction module 207c and the goods and services module 207h.

With each addition of digital tokens 212 to a user's 102 digital wallet 105 in the DL (e.g., blockchain) 110a, the user's net worth on the platform increases. In some aspects, each doner or investor in a player (i.e., each sponsor 107 of a user 102) may become a co-owner of the player or user 102, in that such sponsor 107 may become a fractional agent in a future transfer transaction of that player or user 102 to a hiring club or team that offers him/her an amount of money, e.g., under a sports contract. That is, the sponsor 107 (such as a PM sponsor 107a) may be entitled to a portion of the player's 102 transfer value in the event that a club or team offers the player/user 102 a monetary amount, such as under a sports contract, and such fractional transfer value ownership (which may be tracked by the transaction module 207c and/or the DL 110a, where such fractional allocations may be tracked for the user 102) may be governed by a contract administered through the platform 200 and effected, e.g., via the DL 110a (such as a smart contract entered into by users 102 as a condition to joining the platform 200).

With reference to FIG. 8, in some aspects, the method 300 may further comprise redeeming 318 (such as via the transaction module 207c) the verifier reward amount of points 212 issued to academia-related verifier users 102 for sponsored gifts issued through the platform 200, such as via the goods and services module 207h of the platform 200. The sponsored gifts may be sponsored by one or more of the LR sponsor 107b and/or one or more of the NG sponsor 107c through sponsorship of the platform 200. The sponsorship of the platform may comprise, e.g., a contribution of platform sponsorship digital token(s) 212 to the transaction module 207c of the platform 200. The platform sponsorship digital token(s) 212 may be designated for platform promotions (e.g., rewarding 316 the one or more third party verifiers 103 as the verification reward amount of the digital token(s) 212).

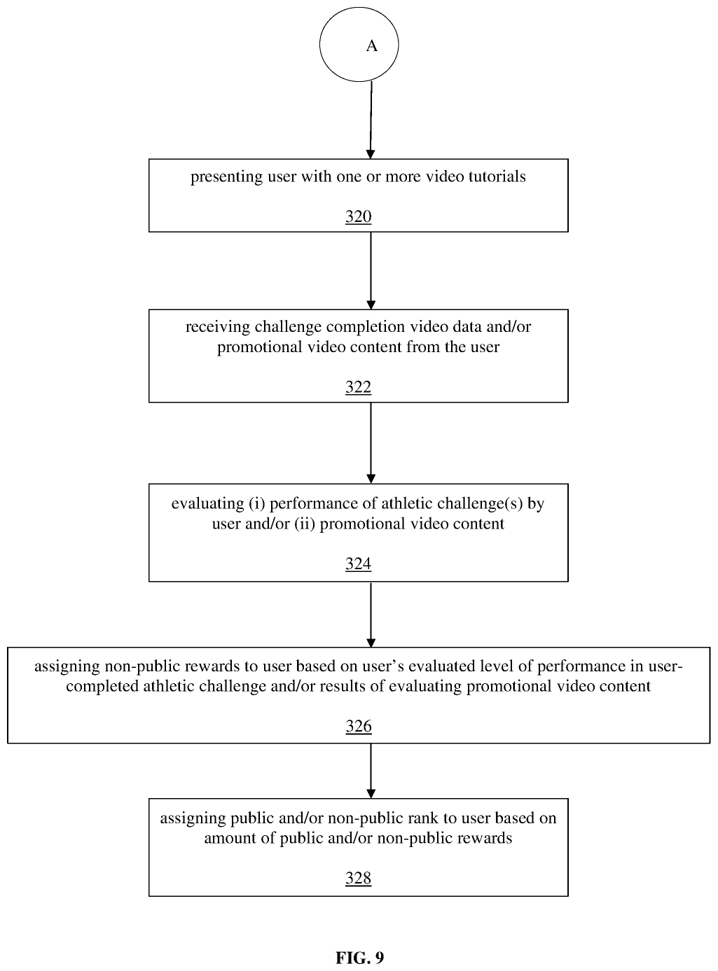

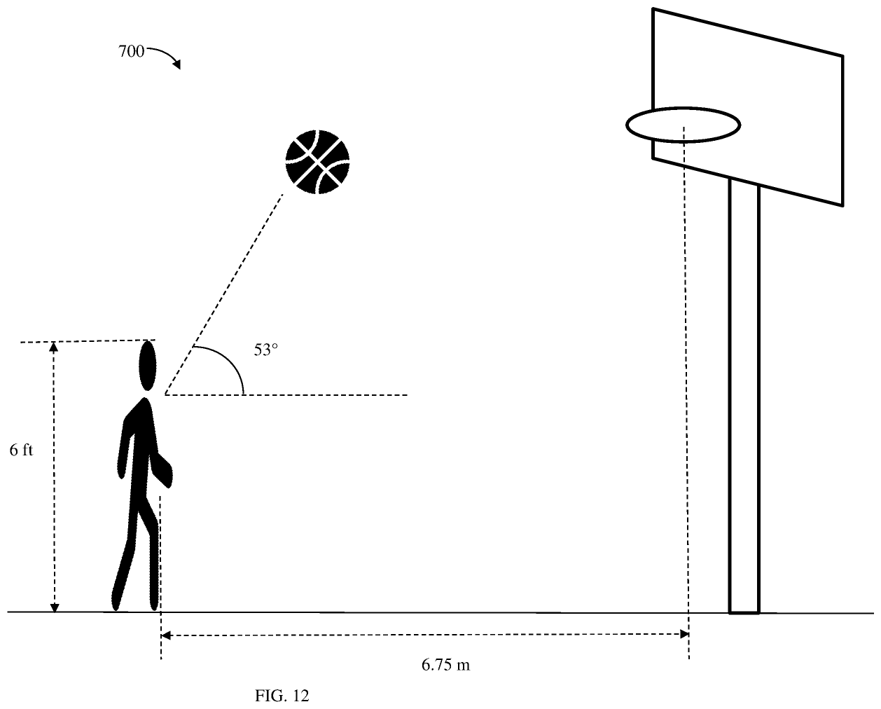

With reference to FIGS. 3 and 9, in some aspects, the method 300 may further comprise: presenting 320 the user 102, such as via a platform computing device 108 of the platform 200, with one or more video tutorials for (i) performing athletic challenge(s) (which may comprise athleticism challenge(s) and/or skills challenge(s)) and/or (ii) creating promotional video content (the promotional content data 506 comprising the promotional video content) promoting the user 102 for recruiting subscribers (i.e., professional sports recruiters (professional users 102)) of the platform 200; receiving 322, via the data intake module 207b, (i) challenge completion video data from the user 102 comprising video footage of a user-completed athletic challenge (the athletic data 504 comprising the challenge completion video data), and/or (ii) the promotional video content from the user comprising video footage promoting the user's athletic abilities; evaluating 324 (i) performance of the athletic challenge(s) by the user 102 (such as by professional sports evaluator(s) 102 or artificial intelligence (AI) and/or machine learning (ML) (such as via the AI module 207g of the platform 200)) and/or (ii) the promotional video content (such as for video quality by, e.g., professional video evaluator(s) 102 or artificial intelligence (AI) and/or machine learning (ML) (such as via the AI module 207g of the platform 200)); and assigning 326, such as via the transaction module 207c, non-public rewards to the user 102, an amount of the assigned non-public rewards 212 (which amount may be determined by the transaction module 207c) based on (i) the user's evaluated level of performance in the user-completed athletic challenge (further non-public rewards 212 may be assigned to the user 102 for any improved or further user-completed athletic challenges, such as that which may be received 322 in further challenge completion video data received from the user 102), and/or (ii) the results of evaluating 324 the promotional video content (e.g., based on an evaluated quality of the promotional video content data). The non-public rewards 212 may comprise, e.g., points 212, such as athleticism points 212 and/or skills points 212 in the case of the evaluated performance of the athletic challenge(s) by the user 102, and/or promotional points 212 in the case of the evaluated promotional video content. The video tutorials may be presented to the users 102 by the platform 200, e.g., weekly (or at some other frequency, such as daily or monthly). With the results of the evaluations of the user-completed athletic challenge(s) and/or of the promotional video content affecting the rankings 602 of the users 102 (as described further, below), the users 102 are expected to be incentivized to attempt to promote themselves through the promotional video content and/or to attempt the challenges presented in the video tutorials, to improve their rankings 602. With reference to FIG. 12, a video tutorial 700 for performing an athletic challenge may comprise, e.g., a basketball-shooting tutorial, demonstrating an optimal angle for a shot from a certain distance (e.g., a 53° release angle for a person standing 6 feet tall and standing back from the middle of the basketball hoop by 6.75 meters).

In some aspects, the method 300 may further comprise generating 334 (such as via the ranking module 207j) a list of the top-ranked users or players 102, and arranging 336 tournaments (such as “Best-in-Class” tournaments) through the platform 200, such as via the goods and services module 207h, to physically engage the top-ranked users 102 in real-world tournaments that are expected to attract professional users 102 (e.g., sports scouts) or other recruiters to watch and potentially recruit users or players 102.

With reference to FIGS. 9, 10 and 11, in some aspects, the method 300 may further comprise assigning 328 a rank 602 to the user 102 based on the amount of the rewards 212, such as via the ranking module 207j of the platform 200. The rank 602 may comprise a non-public rank 602, as shown in FIG. 11 (e.g., based on the amount of the non-public rewards 212 assigned to the user), or a public rank 602, as shown in FIG. 10 (e.g., based on the amount of the public rewards 212 assigned to the user). The public rank 602 may, in some aspects, be visible to all users 102 of the platform 200. The non-public rank 602 may, in some aspects, be visible only to recruiting subscribers (professional users 102) of the platform 200, such as sports scouts and/or subscribers 102 belonging to professional sports clubs. The rank 602 (public or non-public) may serve as an indicator (such as to professional users 102 or recruiters 102) of the user's 102 athletic abilities as compared to other users 102 of the platform 200 playing the same sport(s), and the platform 200 may use artificial intelligence (AI) and/or machine learning (ML) to assign the rank 602 (public or non-public) based on the amount of the rewards 212 (public or non-public) assigned to the user 102, such as via the AI module 207g.

In some aspects, the method 300 may further comprise: receiving 330, such as over the network 106 at the platform computing device 108, a request to generate a digital score card 600 for a user 102 based on the athlete data 500 for the user 102; and generating 332 the digital score card 600, such as via the score card generation module 207i of the platform 200. The score card 600 may be printable so as to resemble a physical sports card, similar to those for professional athletes of different sports that are commonly collected by fans. The score card 600 may comprise at least a portion of the athlete data 500, as described above with respect to FIGS. 10 and 11. In some aspects, any user 102 of the platform 200 may request that a digital score card 600 for a particular user/player 102 be generated. In yet further aspects, only recruiting subscribers (professional users 102) of the platform 200 may view a digital score card 600 comprising a non-public rank 602, non-public rewards 630 and/or non-public assessments 620, as shown on the example digital score card 600 depicted in FIG. 11 (whereas any user 102 may view a digital score card 600 comprising only public information, such as a public rank 602, public rewards 630 and/or public assessments 618, as shown on the example digital score card 600 depicted in FIG. 10). In some aspects, the score card generation module 207i may determine, based on whether the request to generate a digital score card 600 is received 330 from a professional user 102 or a non-professional (i.e., regular or premium account) user 102, whether non-public information (e.g., the non-public rank 602, non-public rewards 630 and/or non-public assessments 620) may be displayed on the digital score card 600 (the score card generation module 207i including such non-public information only if the request to generate a digital score card 600 is received 330 from a professional user 102).

In some aspects, a system 200 for sharing athlete data 500 of users 102 of the system 200 may comprise any of the components described herein for carrying out any of the method steps described herein, including, e.g.: one or more platform computing device(s) 108 and the DL (e.g., blockchain) 110a. Each of the platform computing device(s) 108 may comprise, e.g., non-transient computer-readable memory 112, a communication module 114, and at least one processor 120. The platform computing device(s) 108 may also comprise any of the modules of the platform backend 207 described herein. In one example aspect, the platform computing device(s) 108 may each comprise the transaction module 207c, the data intake module 207b, and the verification module 207e. The processor(s) 120 may be communicatively coupled to the memory 112, the communication module 114, and any of the modules of the platform backend 207 described herein, such as the transaction module 207c, the data intake module 207b, and the verification module 207e. In some aspects, the memory 112 may have stored thereon or therein computer-executable instructions for carrying out any of the method steps described herein, including, e.g.: issuing 302, such as via the transaction module 207c of the platform 200, digital tokens 212 for acquisition by the users 102 (such as over a network 106 via respective user computing devices 104) (as described above, each of the digital tokens 212 may have a monetary value and may be purchasable by the users 102 (such as over the network 106) via the transaction module 207c, or from a third party entity 101 (e.g., cryptocurrency exchanges, such as Coinsquare™ or Coinbase™) that is communicatively coupled (such as over the network 106) to the platform 200, such as to the transaction module 207c, via a third party computing device 104 and the communication module 114); receiving 304, such as via the data intake module 207b, the athlete data 500 from a user 102 of the system 200 (such as over the network 106 via the user computing device 104 and the communication module 114); assigning 306 (such as via the transaction module 207c) public rewards 212 to the user 102 based on the athlete data 500 received (such as via the data intake module 207b) (as described above, the public rewards 212 may be visible to all of the users 102 of the system 200); verifying 310 (such as via the verification module 207e of the platform 200) the athlete data 500 by one or more third party verifiers 103 and/or by cross-reference to verifying data (such as photos or scanned documents) received from the user 102 (such as via the user computing device 104, over the network 106, via the communication module 114 and the data intake module 207b); and sending 312 (such as over the network 106) at least a portion of the verified athlete data 500 to the DL (e.g., blockchain) 110a (such as via the communication module 114). As described above, each of the users 102 of the system 200 may have an associated digital wallet 105 on the DL (e.g., blockchain) 110a for maintaining a balance of one or more of the digital tokens 212.

The platform or system 200 is expected to provide a combination of a marketplace function (to find, rank and select talented sports players across diverse types of sports who are engaged in pre-professional and//or amateur sports) with a computer-implemented infrastructure that will allow investors to help the players 102 by providing financial support using digital tokens 212 (e.g., cryptocurrency) issued through the platform 200 (such as via the transaction module 207c) to be used in the platform 200 ecosystem 100 of partners and services, such as via the transaction module 207c to acquire goods and/or services via the goods and services module 207h, so as to overcome deficiencies in the current sports recruiting landscape, such as limited amateur athlete visibility to scouts and recruiters, non-verified and self-serving data, and disjointed, non-uniform data from disparate sources, as well as the other deficiencies noted above.

It is expected that the platform 200 may provide a scoring utility that uses a customized evaluation algorithm, allowing aspiring players or users 102 to be ranked by their abilities and seen by sports recruiters (i.e. professional users) 102. The challenge completion video data and/or the promotional video content received 322 from users 102 (such as in response to video tutorials 700 presented 320 to the users 102), in which data from one or more sensors or input devices 118 of the user computing device(s) 104 is collected, may be used to train various artificial intelligence and/or machine learning systems (such as may be implemented in the AI module 207g) to improve user rankings 602 and thus, talent selection by professional users/sports recruiters 102. As noted above, the providing of such challenge completion video data and/or promotional video content may be incentivized, such as by assigning 326 non-public rewards 212 to the users 102, to provide further athlete data 500 to the platform 200 by prompting users 102 to collect data 500 which may be lacking in the users' profiles and/or to appropriately characterize or label athlete data 500 (as may be determined by the AI module 207g). Scoring may be based on the tokens 212 so that every step has an associated timestamp, transaction log, etc., stored in the DL 110a, which may bring more transparency and credibility to the scoring mechanism.

In some aspects, rewards 212 that comprise points 212 cannot be bought, sold or transferred on the platform 200, and comprise no monetary value. In some aspects, visibility of a user 102 on the platform 200 may correlate to the amount of points 212 the user 102 has accumulated, with greater visibility (such as by being closer to the top of search results when searched by a professional user 102 by, for example, a search module 207k of the platform 200) resulting from a greater amount of accumulated points 212.

It is expected that the modularization of the various functionalities described herein, such as via the various processing modules 205 described herein (including, e.g., one or more of the login verification module 207a, data intake module 207b, transaction module 207c, service provider accreditation module 207d, verification module 207e, extraction/reporting module 207f, artificial intelligence and/or machine learning (AI) module 207g, goods and services module 207h, score card generation module 207i, ranking module 207j, and/or search module 207k), may allow for improved performance and performance management versus a system in which such functionalities are not modularized as described herein, such as by allowing for: (i) computer processing resources (e.g., the processor(s) 120) to be distributed as required to only those modules 205 of the platform backend 207 requiring such resources at any point in time, and/or by allowing for processing resources (e.g., the processor(s) 120) to be added to the system 200, e.g., for any given module(s) 205 that may require additional processing resources (e.g., the processor(s) 120), as may be determined by monitoring of performance metrics of the platform or system 200.

It will be appreciated that the presently described aspects for acquiring, verifying, storing, and sharing athlete data using rewards-based incentives for data sharing and verification may require large data storage capacity via the database(s) 110 and distributed ledger(s) 110a, particularly in view of the storage capacity required to store copies, and in some aspects, backups, of uploaded video submissions from users, each of which may be comprise, e.g., 500 MB of storage space.

In some aspects, a digital signature of all DL data 208 may be stored on the DL 110a, and only this hash may be recorded to guarantee authenticity of the data to which it refers. In yet further aspects, there may also be a digital signature to ensure who is responsible for recording the data on the DL 110a. A digital signature is used to guarantee that a known source generated the message (non-repudiation), and that the message was not altered in transit (integrity). A hash is used to verify message integrity; i.e., if a message changes, the hash of the message will also change.

The presently described data-sharing aspects may use a “universal” data-sharing format derived by, e.g., a consortium of parties (such as a standardized data format for data shared in the manners described herein). In some aspects, e.g., the standard format used to exchange information or data may comprise JSON, although the information exchange standard may be substituted with a binary, faster and programming language-agnostic approach, like gRPC, where data can be exchanged between different types of platforms and computer languages.

The presently described aspects are expected to add value to user-provided data through validation, including that data which is provided by any type of user 102 described herein (e.g., athletes, parents, teachers, coaches, sponsors, etc.). In some aspects, appropriate controls would be in place to verify data provided by young users (e.g., under the age of 18) who do not have a credit card, such as an internal team that observes specified security and privacy criteria (e.g., the Brazilian General Data Protection Law (LGPD)). In some aspects, the data provided by users may be encrypted for storage, and redundant backups of the provided and/or generated data may be maintained, such as in remote data storage, with appropriate processes in place for access control, data restoration and disaster recovery. In some aspects, third-party systems employing advanced data access and recovery mechanisms (e.g., facial recognition) may be used in place of a back-office team.

In some aspects, as an alternative to validation, Terms of Use may be required to be agreed to by users 102 as a condition for accessing the platform 200, requiring each user 102 to assume responsibility for the correctness of any data provided by the user and to ensure that such data complies with all applicable laws, for example.

Since the professional UI 204 may be accessed by sports associations or clubs, medical professionals or other sports or athletic performance authorities or partners of the platform 200, it will be appreciated that different forms of documentation may be required from each type of such professional users 102 in order to validate such professionals.

Some value propositions for the presently described aspects are described below.

Social Finance (SoFi)

It is expected that the DL 110a of the platform 200 may be used to effect payments for social networks (Social Finance (SoFi)). The platform 200 may comprise a SoFi toolkit of decentralized financial primitives that are optimized to support young athletes, which is expected to encourage the practice of sports, well-being and visibility of young athletes while promoting integration between sports professionals. The presently described social financial investment platform with a focus on young athletes from all over the world and across a variety of sports is expected to provide a holistic view of the world of social finance in sports. It is expected that the presently described DL, e.g., blockchain 110a, may revolutionize social payments on a large scale. The presently described transaction module 207c may allow the platform 200 to operate as a new type of bank that does not lend out anyone's money.

The presently described aspects may also provide for a news feed platform for stories related to sports separated by segments (e.g., football, basketball, etc.), and in addition to ‘share’ and ‘favourite’ buttons or functions, the platform 200 may offer the following options.

Awards

Supporting young athletes in the early stages of their careers allows users to invest both in the sport and in the athlete's personal development. Awards may comprise virtual gifts that have an intrinsic value that can be used to buy sports goods. To encourage their progress in a sport, athletes can receive donations on their profile or in text, image, or video posts. Through a donation button, a donor can choose a virtual gift, represented by a distinct seal, and then make the donation through a post. Each seal may be purchased for a price and traded only for a specific token amount. If the donor does not have sufficient funds in tokens 212 in his wallet 105 to purchase the virtual gift, he must convert another currency, such as fiat currency or cryptocurrency, to the required amount of tokens 212, such as via the transaction module 207c. Such awards may be given by any user that wants to stimulate young athletes without having any major financial investments. By giving such awards to athlete users 102, it is possible to encourage and motivate athletes to improve their skills. Once obtained, these virtual gifts can be converted into credits or points. Such awards may comprise, e.g., non-fungible tokens or NFTs, such as images and/or written depictions of messages including, e.g., “Team player”, “Leadership”, “Coach”, “Courage”, “Optimism”, “Inclusive”, “Awesome”, “Thank you”, “Achiever”, “Problem solver”, “Kind heart”, and “Creative”.

NFTs

In some aspects there may be an option to create on-demand a non-fungible token (NFT) from a selected post or an athlete profile. The platform 200 may upload the publication to obtain the signature of the file and the creation of the token related to this unique key which will be immediately associated with the user account. The values of this token will be determined using its own algorithm or based on a value to be defined by the user or by the person responsible for the post. Every NFT token may have in its smart contract a commission amount to be transferred to the asset creator and to the platform 200 administrator in perpetuity at each transaction. All NFT minted in the platform 200 may keep a perpetual royalty reward for the athlete and for the platform 200 administrator.

In some aspects, the platform 200 may comprise an NFT marketplace to, e.g., raise funds to donate inside the platform 200 to athletes 102 and offer its NFT to the secondary market to subsidize its costs to achieve professional sports levels. Such NFTs may include, e.g., score cards 600, video footage of the user's athleticism, videos and photos posted on the social platform, virtual t-shirts, and a team pack.

Platform Shop

The platform 200 administrator could buy wholesale and sell through tokens 212 or donations items such as vests, balls, books, soccer shoes, and other sports goods and/or services, as described above. In some aspects, the platform 200 may integrate with one or more partner store(s), making it possible to sell on-demand through a white-label platform using tokens 212.

Platform Academy

As described above, the platform 200 may provide digital video guides, courses, and the like, with sports experts (payable via tokens 212), thus providing a digital academy for on-demand access by users 102 when needed.

Validators

The data validation system used within the DL (e.g., blockchain) may combine the number of confirmations, the credibility of the auditor and other strategies to confirm the authenticity of the information. This validation process creates a cryptographically-secured, time-locked ledger that can then be used to extract data. The platform 200 is designed to ensure that validators are always motivated to verify information, gaining rewards in the form of cryptocurrency tokens 212. The tokens are sent straight to the validator's wallet 105. These token rewards 212 can be traded with other cryptocurrencies on a cryptocurrency trading platform, as described above.

The platform 200 is expected to provide a decentralized, trustworthy data storage system in which validators can endorse transactions to earn a reward 212. The platform 200 is designed to be a decentralized, peer-to-peer, DL (e.g., blockchain)-based platform that provides a self-regulated environment where anyone can contribute and share information. The platform 200 is expected to encourage the production of original content and rewards actors for providing reliable information. The platform 200 has a hybrid validation model which combines a strong, robust, and costly validation carried out by special nodes (miners), with a precise, punctual, and cheap validation carried out by all users (validators).

While the platform 200 may use only platform-accredited validators to audit submitted data (e.g., video of users with completed challenges), it is expected that the platform 200 could evolve to a more decentralized, community-oriented validation model, where more validators participate in the validations. In this way, each data can have its credibility audited by the community and the value of the data related to the number of validations and the weight of each validator, whether authorities such as coaches, educational institutions, sports teams, or sports associations (e.g.: 10 validations from other athletes, 3 validations from trainers, 2 validations from internal staff) further enhancing the quality of stored data and creating a consensus mechanism for validation. The rewards value may be calculated based on the level of verification of the data (e.g., by professional verifier users vs. non-professional verifier users), for example, and/or any other relevant factors.

Such validation participation may be motivated by rewards offered as specific tokens 212 and following a distribution algorithm (and such functionality may resemble the role of miners in blockchain networks).

An example of distribution of “ad token” Tokens is shown below:

| | |
|---|---|
| 1°. Verified-User (e.g. Coach) = 1 | 1°. Athlete = 0.1 “ad token”; |
| “ad token”; | |
| 2°. Verified-User (e.g. Coach) = 0.5 | 2°. Athlete = 0.05 “ad token”; |
| “ad token”; | |
| 3°. Verified-User (e.g. Coach) = 0.25 | 3°. Athlete = 0.025 “ad token”; |
| “ad token”; | |
| 4°. Verified-User (e.g. Coach) = 0.125 | 4°. Athlete = 0.0125 “ad token”; |
| “ad token”; | |

Following the example above we can see that the first coach to validate the data will receive 1 “ad token”, the second 0.5, the third 0.25, the fourth 0.125 and so on. In the case of validations by common users, such common or non-professional verifier users may receive virtual badges, as described above. With such a system of validation rewards distribution, even after thousands of validations, no more than two tokens 212 will be distributed.

To become an accredited validator, a Know Your Customer (KYC) process may be used in order to accredit the validator by the platform 200 (such as via the accreditation module 207d), and a monthly fee, that can be deducted using “ad tokens”, may be required.

Some advantages of being a validator may include, e.g.:
having the monthly fee exempt after a certain number of validations;
revenue generation through token validations;
first-hand access to athlete data; and
listing in the partner network.

Play to Earn Platform

The platform 200 may comprise a play-to-earn function, which is a unique gaming model that rewards athlete users 102 for playing games and completing challenges (e.g., a message from the platform 200 to such users 102 may be “Challenge yourself while also helping to build your collection of custom NFT trophies to show your status”). The platform 200 may use the above-described coach mechanism to validate data provided by users 102. Some other ways to get rewarded are receiving custom rewards, digital goods, virtual tokens, cryptocurrency or NFTs, or real-world prizes, for example.

Platform Dream Team

The platform 200 may receive sponsorships (e.g., monthly) from advertisers who advertise on the platform 200, as described above. Sponsorships paid in fiat currency may automatically be converted into cryptocurrency to be distributed among certain players who stand out in a given period according to previously stipulated criteria (e.g., the top 5 players who fulfilled the greatest number of challenges within the platform within a month earn 1/10 of the advertising or sponsorship value each, and the remainder of the advertising or sponsorship value goes to the platform 200 administrator).

Battle Card

Flip Cards or score cards 600 may comprise an original collection of NFT trading cards featuring elite-level young players 102 from a variety of sports ranked by the platform 200 algorithm, as described above. The cards 600 may be released on demand. Each of such NFT flipping cards may be tradable within the Flip Cards digital ecosystem. Flip Cards may also allow players 102 to battle an opponent in a turn-based battle. Each player has different stats which are key to their success in the game. Such NFT flip cards may also be available for purchase on the secondary market, which provides an excellent opportunity to build a collection of such cards before the players “go pro” or become professional athletes.

Patreon Module

The purpose of Patreon is to help support and reward young and aspiring athletes and also to provide aid in the form of sports goods, food and education. Payments may be made in a transparent way through the use of a DL (e.g., blockchain) transaction and donations recursively.

The Patreon Module is a way of giving small amounts of money regularly to fund someone. It is especially important in the world of sports to give children the opportunity to partake in the sport that they love, particularly those from underprivileged backgrounds and who might not otherwise be able to afford an education in the sport, but through the Patreon module it is expected that such users 102 may receive funds to facilitate the pursuit of their athletic aspirations.

Sponsorship is an important financial support system that allows young athletes to dedicate their time and energy to their sport. The Patreon module provides a micro-sponsorship platform within the platform 200 to help those who need some support in order to pursue their sport(s) of interest. The Patreon module is expected to provide an affordable and convenient way to support and educate children and athletes 102, and is expected to be more effective than other forms of donations outside of the platform 200. In some aspects, the Patreon module may provide for crowdfunding with open-end payments. The Patreon module is expected to allow people to help the lives of young athletes 102 by providing them with financial assistance for food and school or giving them resources to buy sports equipment or to pay for training, for example.

All payments via the Patreon module may be made in a transparent way using DL (e.g., blockchain) transactions, which is also expected to make such payments secure. The platform 200 may, in some aspects, be responsible for the custody of the cryptocurrency value that is donated via the Patreon platform (such as via the transaction module 207c), realizing the transactions monthly to the athlete(s) 102.

Crypto Exchange/Fantasy Sports

Each of the users 102 of the platform 200 has an associated digital wallet 105 on the DL (e.g., blockchain) 110a for maintaining a balance of one or more of the digital tokens 212, as described above. The platform tokens 212 may be minted by the DL (e.g., blockchain) 110a of the platform 200, and all on-platform voting may be executed as a series of smart contracts which run through the DL (e.g., blockchain) 110a. In some aspects, fans may be able to purchase branded tokens 212 from some of the better-ranked and most popular sports athletes 102 on the platform 200. The tokens 212 may allow users 102 to influence their athletes 102 via popular vote and become eligible for rewards and recognition.

The DL-based voting system is innovative in that it utilizes the DL (e.g., blockchain) to keep the voting system secure. The blockchain (as an example of a DLT) is a distributed ledger system that can record transactions between multiple computers with no centralized authority. Voters may utilize the purchase of tokens 212 to exercise influence over athlete(s) 102. Fans may vote with their tokens 212 to determine the winners of games, races, and other events. Fans who own more tokens 212 may have greater voting power than those with fewer tokens 212.

It will be appreciated that in a centralized cryptocurrency exchange model, all users 102 may be paired with tokens 212 (if they have a balance of tokens 212). It will further be appreciated that in a decentralized cryptocurrency exchange model, the platform 200 may interface with a network of businesses that accept the tokens 212.

Fans or users 102 of the platform 200 may purchase tokens 212 for various reasons, such as those described above, including, for example:
influencing club/athlete decisions;
earning virtual VIP rewards (NFTs) and experiences;
trading fan tokens;
receiving exclusive promotions;
participating in score prediction games;
participating in the Fantasy Exchange platform; and
participating in games, surveys and more.

In some aspects, it may be possible to convert such credits into investment player tokens (professional income share agreements).

Advertising System

Tokens 212 may be spent on advertising using the platform's network of ads, allowing more relevant customers to be reached within a particular advertising budget and helping to improve the lives of many young athletes 102 of the platform 200.

Premium Account Features

Premium accounts may be associated with special features, which may incentivize users to opt for a premium account. For example, some reasons why users may opt for a premium account may include:
Expand video profile time, custom icons, background;
Participation in cryptocurrency airdrops;
Navigation without advertising;
Exclusive avatar clothes and items;
Membership Hall;
Premium Awards;
Coins bonus as a welcome gift (e.g., 1000 tokens); and
Annual or monthly plans.

Partner Network

The wallets 105 may be added to the whitelists of the platform's smart contracts, allowing merchants to receive the platform currency or tokens 212. Such third party companies may be accredited, such as if they respect certain criteria to benefit young athletes 102, and if accredited, such companies may be part of a guide for companies and professionals in a specific application module. A monthly fee may be required to be paid, which may be converted into advertising tokens (“ad tokens”) 212. These tokens 212 can be used to sponsor specific athletes or donated to the ranking system pool that distributes the values monthly.

Score Card

The above-described score cards 600 may comprise printable virtual cards 600. Each card 600 may have a QR code graphic that may allow for the use of credits on a platform partner network, and which in some aspects may be valid until a particular date.

Blockchain Oracle

A blockchain oracle is a third-party service that connects smart contracts with the outside world, primarily to provide information from the world, but also the other way around (i.e., from the platform 200 to the world). The world's information encapsulates multiple sources so that decentralized knowledge may be obtained.

Sharing athlete data 500 of users 102 of the platform 200, the platform 200 will be one oracle accessible through its own smart contract accessible through its own app. For each query/transaction made by smart contracts via API (e.g., web3), a small fee may be charged by the platform 200 (such as via the transaction module 207c).

Other Possibilities

Other value propositions for the presently described aspects may include, for example:
Experience—a video record from the athlete bought with tokens 212;
Tickets—any user can promote their games by selling NFT tickets;
Betting—a digital betting system;
Events—Calendar of amateur competitions and events;
Jobs—Validated list of sports professionals (school, teachers, doctors, etc.) and related job postings;
Meta—A 3D avatar of the athlete; and
Smart Watch integration.

It is expected that the platform 200 may address the present scarcity and reliability in amateur athletic data, by providing an alternative source of athlete data which is expected to provide scouts and analysts with access to consistent and reliable information of millions of players 102, to facilitate the discovery of talented players 102 from a large pool of players.

The platform is expected to allow for various revenue streams, including, but not limited to, revenue through brand sponsorship, media sponsorship, premium subscriptions by premium users 102, and micro-sponsorship, as well as the various other forms of revenue described above.

Unless otherwise explained, all technical and scientific terms used herein have the same meaning as commonly understood by one of ordinary skill in the art to which this disclosure belongs. Although any methods and materials similar or equivalent to those described herein can be used in the practice for testing of the present invention, the typical materials and methods are described herein.

It is also to be understood that the terminology used herein is for the purpose of describing particular aspects only and is not intended to be limiting. Any patent applications, patents, and publications cited herein are to assist in understanding the aspects described. All such references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers, and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention.

It will be understood that any component defined herein as being included may be explicitly excluded from the claimed invention by way of proviso or negative limitation. In addition, all ranges given herein include the end of the ranges and also any intermediate range points or values within the stated ranges, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Any method steps described herein may be performed in any suitable sequence or arrangement, despite that one particular example sequence or arrangement has been shown and/or described herein.

The abbreviation, “e.g.” is derived from the Latin phrase exempli gratia, and is used herein to indicate a non-limiting example. Thus, the abbreviation “e.g.” is synonymous with the term “for example.” The word “or” is intended to include “and” unless the context clearly indicates otherwise.

What is claimed is:

1. A computer-implemented method for sharing, on a computer-implemented data-sharing platform, athlete data of users of the platform, the method comprising:
issuing, via a transaction module of the platform, digital tokens for acquisition by the users of the platform, over a network via a user computing device, each of the digital tokens having a monetary value and purchasable by the users over the network via the transaction module or from a third party entity communicatively coupled over the network to the transaction module via a third party computing device;
receiving, via a data intake module of the platform, the athlete data from a user of the platform via the user computing device over the network;
assigning, via the transaction module, public rewards to the user based on the athlete data received, the public rewards visible to all of the users of the platform;
verifying, via a verification module of the platform, the athlete data by one or more third party verifiers and/or by cross-reference to verifying data received from the user via the data intake module; and
sending, over the network, at least a portion of the verified athlete data to a distributed ledger, the platform comprising the distributed ledger;
wherein each of the users of the platform has an associated digital wallet on the distributed ledger for maintaining a balance of one or more of the digital tokens.

2. The computer-implemented method of claim 1, wherein the data intake module comprises a data input interface for the receiving of the athlete data via input(s) of the user, via the user computing device, to the data input interface.

3. The computer-implemented method of claim 1, wherein the distributed ledger comprises a blockchain and wherein the sending the at least the portion of the verified athlete data to the distributed ledger comprises validating the at least the portion of the verified athlete data as a block transaction on the blockchain by a proof of stake validation.

4. The computer-implemented method of claim 1, wherein the public rewards comprises: (i) points; (ii) virtual badges of ascending levels corresponding to respective ascending amounts of the points; (iii) or both;
and wherein the assigning, by the transaction module, the public rewards based on the athlete data received via the data intake module comprises adjusting an amount of the assigned public rewards to be commensurate with an amount of the athlete data received via the data intake module and/or an amount of the types of the athlete data received via the data intake module.

5. The computer-implemented method of claim 1, wherein the digital tokens are redeemable by the user (i) via the third party entity or the transaction module to realize the monetary value as fiat currency and/or cryptocurrency; and/or (ii) via the transaction module, to upgrade an account level of the user and/or to purchase goods and/or services offered on the platform via a goods and services module of the platform.

6. The computer-implemented method of claim 1, wherein the athlete data comprises: (i) non-athletic data; (ii) athletic data comprising at least athleticism data and skills data; and/or (iii) promotional content data.

7. The computer-implemented method of claim 6 wherein:
the non-athletic data comprises name, nickname, birthday, gender, sport(s) played, position(s) played, laterality, associated sports organizations, anthropometric data, and/or a list of and contact information for the one or more third party verifiers;
the athleticism data comprises user self-assessment(s) of speed, quickness, agility, strength, and/or endurance;
the skills data comprises user self-assessment(s) of skill(s) related to sport(s) played by the user; and
the promotional content data comprises video footage of the user's athletic achievements.

8. The computer-implemented method of claim 7, wherein the user acquires the one or more of the digital tokens by: (i) purchasing the one or more of the digital tokens from the third party entity or the transaction module; (ii) receiving the one or more of the digital tokens from one or more sponsors via the transaction module; and/or (iii) earning, via the transaction module, the one or more of the digital tokens through the sending the at least the portion of the verified athlete data to the distributed ledger, the one or more digital tokens acquired by the user comprising a user verification reward amount of the digital tokens determined by the transaction module.

9. The computer-implemented method of claim 8, wherein the at least the portion of the verified athlete data that is sent to the distributed ledger comprises the athletic data and/or the promotional content data.

10. The computer-implemented method of claim 9, wherein each of the one or more sponsors comprises one of:
(i) a personal or micro (PM) sponsor, each said PM sponsor designating less than a first threshold amount of the digital tokens to one or more PM-sponsored users of the platform;
(ii) a local or regional (LR) sponsor, each said LR sponsor designating between the first threshold amount and a second threshold amount of the digital tokens to sponsoring: (A) the platform, (B) one or more LR-sponsored users of the platform, and/or (C) one or more LR-sponsored tournaments associated with the platform; and
(iii) a national or global (NG) sponsor, each said NG sponsor designating more than the second threshold amount of digital tokens to sponsoring: (A) the platform, (B) one or more NG-sponsored users of the platform, and/or (C) one or more NG-sponsored tournaments associated with the platform.

11. The computer-implemented method of claim 10, further comprising: incentivizing, via the transaction module, the verifying the athlete data by the one or more third party verifiers by rewarding the one or more third party verifiers for the verifying the athlete data with a verifier reward.

12. The computer-implemented method of claim 11, wherein the verifier reward comprises (i) a verifier reward amount of the digital tokens determined by the transaction module and issued to professional verifier users of the platform by the transaction module, and/or (ii) a verifier reward amount of points determined by the transaction module and issued to non-professional verifier users of the platform by the transaction module;
wherein each of the professional verifier users comprises a service provider accredited by a service provider accreditation module of the platform as a professional service provider;
and wherein each of the non-professional verifier users is not accredited by the service provider accreditation module for providing professional services.

13. The computer-implemented method of claim 12, wherein the non-professional verifier users comprise peers of the user, academia-related verifier users of the platform, and/or other references provided by the user over the network via the user computing device.

14. The computer-implemented method of claim 13, wherein the verifier reward amount of the points issued to the academia-related verifier users is redeemable via the transaction module for sponsored gifts issued through the platform via a goods and services module of the platform.

15. The computer-implemented method of claim 14, wherein the sponsored gifts are sponsored by one or more of the LR sponsor and/or one or more of the NG sponsor through sponsorship of the platform, the sponsorship of the platform comprising a contribution of platform sponsorship digital token(s) to the transaction module of the platform.

16. The computer-implemented method of claim 15, further comprising:
presenting the user, via a platform computing device of the platform, with one or more video tutorials for (i) performing athletic challenge(s) and/or (ii) creating promotional video content promoting the user for recruiting subscribers of the platform;
receiving, via the data intake module, (i) challenge completion video data from the user comprising video footage of a user-completed athletic challenge, the athletic data comprising the challenge completion video data, and/or (ii) the promotional video content from the user comprising video footage promoting the user's athletic abilities, the promotional content data comprising the promotional video content;
evaluating, by artificial intelligence (AI) and/or machine learning (ML) via an AI module of the platform, (i) performance of the athletic challenge(s) by the user, and/or (ii) the promotional video content; and
assigning, via the transaction module, non-public rewards to the user, an amount of the assigned non-public rewards determined by the transaction module and based on (i) the user's evaluated level of performance in the user-completed athletic challenge, and/or (ii) results of the evaluating the promotional video content.

17. The computer-implemented method of claim 16, further comprising:
assigning, via a ranking module of the platform, a public rank to the user based on an amount of the assigned public rewards, the public rank visible to all users of the platform; and/or
assigning, via the ranking module, a non-public rank to the user based on the amount of the assigned non-public rewards, the non-public rank visible to recruiting subscribers of the platform.

18. The computer-implemented method of claim 17, further comprising:
receiving, over the network at the platform computing device, a request to generate a digital score card based on the athlete data; and
generating, via a score card generation module of the platform, the digital score card, the score card comprising at least a portion of the athlete data.

19. The computer-implemented method of claim 17, further comprising:
generating, via the ranking module, a list of top-ranked users; and
arranging, via the goods and services module, tournaments for the top-ranked users.

20. A system for sharing athlete data of users of the system, the system comprising:
one or more platform computing device(s), each of the platform computing device(s) comprising non-transient computer-readable memory, a communication module, a transaction module, a data intake module, a verification module, and at least one processor communicatively coupled to the memory, the communication module, the transaction module, the data intake module, and the verification module; and
a distributed ledger;
wherein the memory has stored thereon or therein computer-executable instructions for:
issuing, via the transaction module, digital tokens for acquisition by the users over a network via respective user computing devices, each of the digital tokens having a monetary value and purchasable by the users over the network via the transaction module or from a third party entity communicatively coupled over the network to the transaction module via a third party computing device and the communication module;
receiving, via the data intake module, the athlete data from a user of the system, over the network via a user computing device and the communication module;
assigning, via the transaction module, public rewards to the user based on the athlete data received, the public rewards visible to all of the users of the system;
verifying, via the verification module, the athlete data by one or more third party verifiers and/or by cross-reference to verifying data received from the user via the user computing device, over the network, via the communication module and the data intake module; and
sending, over the network, at least a portion of the verified athlete data to the distributed ledger via the communication module;
wherein each of the users of the system has an associated digital wallet on the distributed ledger for maintaining a balance of one or more of the digital tokens.

21. The system of claim 20, wherein the distributed ledger comprises a blockchain and wherein the sending the at least the portion of the verified athlete data to the distributed ledger comprises validating the at least the portion of the verified athlete data as a block transaction on the blockchain.

22. The system of claim 20, wherein the public rewards comprises: (i) points; (ii) virtual badges of ascending levels corresponding to respective ascending amounts of the points; (iii) or both;
and wherein the assigning, by the transaction module, the public rewards based on the athlete data received via the data intake module comprises adjusting an amount of the assigned public rewards to be commensurate with an amount of the athlete data received via the data intake module and/or an amount of the types of the athlete data received via the data intake module.

23. The system of claim 20, wherein the digital tokens are redeemable by the user (i) via the third party entity or the transaction module to realize the monetary value as fiat currency and/or cryptocurrency; and/or (ii) via the transaction module, to upgrade an account level of the user and/or to purchase goods and/or services offered on the platform via a goods and services module of the platform.

24. The system of claim 20, wherein the athlete data comprises: (i) non-athletic data; (ii) athletic data comprising at least athleticism data and skills data; and/or (iii) promotional content data.

25. The system of claim 24, wherein:
the non-athletic data comprises name, nickname, birthday, gender, sport(s) played, position(s) played, laterality, associated sports organizations, anthropometric data, and/or a list of and contact information for the one or more third party verifiers;
the athleticism data comprises user self-assessment(s) of speed, quickness, agility, strength, and/or endurance;
the skills data comprises user self-assessment(s) of skill(s) related to sport(s) played by the user; and
the promotional content data comprises video footage of the user's athletic achievements.

26. The system of claim 25, wherein the user acquires the one or more of the digital tokens by: (i) purchasing the one or more of the digital tokens from the third party entity or the transaction module; (ii) receiving the one or more of the digital tokens from one or more sponsors via the transaction module; and/or (iii) earning, via the transaction module, the one or more of the digital tokens through the sending the at least the portion of the verified athlete data to the distributed ledger, the one or more digital tokens acquired by the user comprising a user verification reward amount of the digital tokens determined by the transaction module.

27. The system of claim 26, wherein the at least the portion of the verified athlete data that is sent to the distributed ledger comprises the athletic data and/or the promotional content data.

28. The system of claim 26, wherein each of the one or more sponsors comprises one of:
(i) a personal or micro (PM) sponsor, each said PM sponsor designating less than a first threshold amount of the digital tokens to one or more PM-sponsored users of the platform;
(ii) a local or regional (LR) sponsor, each said LR sponsor designating between the first threshold amount and a second threshold amount of the digital tokens to sponsoring: (A) the platform, (B) one or more LR-sponsored users of the platform, and/or (C) one or more LR-sponsored tournaments associated with the platform; and
(iii) a national or global (NG) sponsor, each said NG sponsor designating more than the second threshold amount of digital tokens to sponsoring: (A) the platform, (B) one or more NG-sponsored users of the platform, and/or (C) one or more NG-sponsored tournaments associated with the platform.

29. The system of claim 28, wherein the transaction module rewards the one or more third party verifiers for the verifying the athlete data with a verifier reward.

30. The system of claim 29, wherein the verifier reward comprises (i) a verifier reward amount of the digital tokens determined by the transaction module and issued to professional verifier users of the platform by the transaction module, and/or (ii) a verifier reward amount of points determined by the transaction module and issued to non-professional verifier users of the platform by the transaction module;
wherein each of the professional verifier users comprises a service provider accredited by a service provider accreditation module of the platform as a professional service provider;
and wherein each of the non-professional verifier users is not accredited by the service provider accreditation module for providing professional services.

31. The system of claim 30, wherein the non-professional verifier users comprise peers of the user, academia-related verifier users of the platform, and/or other references provided by the user over the network via the user computing device.

32. The system of claim 31, wherein the verifier reward amount of the points issued to the academia-related verifier users is redeemable via the transaction module for sponsored gifts issued through the platform via a goods and services module of the platform.

33. The system of claim 32, wherein the sponsored gifts are sponsored by one or more of the LR sponsor and/or one or more of the NG sponsor through sponsorship of the platform, the sponsorship of the platform comprising a contribution of platform sponsorship digital token(s) to the transaction module of the platform.

34. The system of claim 33, wherein:
a platform computing device of the platform presents the user with one or more video tutorials for (i) performing athletic challenge(s) and/or (ii) creating promotional video content promoting the user for recruiting subscribers of the platform;
the data intake module receives (i) challenge completion video data from the user comprising video footage of a user-completed athletic challenge, the athletic data comprising the challenge completion video data, and/or (ii) the promotional video content from the user comprising video footage promoting the user's athletic abilities, the promotional content data comprising the promotional video content;
an artificial intelligence (AI) and/or machine learning (ML) module of the platform evaluates (i) performance of the athletic challenge(s) by the user, and/or (ii) the promotional video content; and
the transaction module assigns non-public rewards to the user, an amount of the assigned non-public rewards determined by the transaction module and based on (i) the user's evaluated level of performance in the user-completed athletic challenge, and/or (ii) results of the evaluating the promotional video content.

35. The system of claim 34, further comprising:
a ranking module of the platform assigns a public rank to the user based on an amount of the assigned public rewards, the public rank visible to all users of the platform; and/or
the ranking module assigns a non-public rank to the user based on the amount of the assigned non-public rewards, the non-public rank visible to recruiting subscribers of the platform.

36. The system of claim 35, wherein:
the platform computing device receives a request to generate a digital score card based on the athlete data over a network; and
a score card generation module of the platform generates the digital score card, the score card comprising at least a portion of the athlete data.

37. The system of claim 35 further comprising:
the ranking module generates a list of top-ranked users; and
the goods and services module arranges tournaments for the top-ranked users.
